(19) United States
(12) Patent Application Publication
(10) Pub. No.: US 2025/0228939 A1
(43) Pub. Date: Jul. 17, 2025

(54) ENHANCED IMMUNE CELL THERAPY

(71) Applicant: LYELL IMMUNOPHARMA, INC., South San Francisco, CA (US)

(72) Inventor: Bijan A. BOLDAJIPOUR, San Francisco, CA (US)

(21) Appl. No.: 18/703,185

(22) PCT Filed: Oct. 20, 2022
(86) PCT No.: PCT/US2022/078444

§ 371 Date: Apr. 19, 2024

Related U.S. Application Data
(60) Provisional application No. 63/270,523, filed on Oct. 21, 2021.

(51) Int. Cl. A61K 40/11; A61K 9/00; A61K 40/31; A61K 40/32; A61P 35/00; C12N 5/0783; C12N 9/22; C12N 15/11

(52) U.S. Cl. CPC A61K 40/11; A61K 9/0019; A61K 40/31; A61K 40/32; A61P 35/00; C12N 5/0636; C12N 9/22; C12N 15/11; A61K 2239/31; C12N 2310/20; C12N 2510/00

(57) ABSTRACT

The present disclosure provides engineered human cells (e.g., T cells) for treatment. Also provided are expression constructs for making the engineered cells.

### CROSS-REFERENCE TO RELATED APPLICATION

This application claims priority from U.S. Provisional Application No. 63/270,523, filed Oct. 21, 2021, the disclosure of which is incorporated herein by reference in its entirety.

### SEQUENCE LISTING

The instant application contains a Sequence Listing that has been submitted electronically in XML format and is hereby incorporated by reference in its entirety. Said XML copy, created on Oct. 5, 2022, is named 026225_WO019_SL.xml and is 2,485 bytes in size.

### BACKGROUND OF THE INVENTION

Adoptive T cell therapy has been studied intensively in recent years as a potential venue for cancer treatment. In many studies, therapeutic T cells are engineered to express an antigen receptor specific for a tumor antigen. However, one challenge facing T cell therapy is the lack of persistence of T cells in vivo due to a phenomenon known as T cell exhaustion. (See, e.g., Fraietta et al., Nat Med. (2018) 24 (5): 563-71; Long et al., Nat Med. (2015) 21 (6): 581-90; and Eyquem et al., Nature (2017) 543 (7643): 113-7). T cell exhaustion is characterized by marked changes in metabolic function, transcriptional reprogramming, loss of effector functions (e.g., reduced cytokine secretion and cytotoxicity), increased expression of multiple surface inhibitory receptors, and apoptosis. T cell exhaustion has been attributed to constant antigen exposure, leading to continuous TCR signaling, or to tonic antigen-independent signaling through an engineered antigen receptor on T cells (see, e.g., Long, supra). Prevention or reversal of T cell exhaustion has been sought as a means to enhance T cell effectiveness, e.g., in patients with cancer or chronic infections and in T cell therapy. See, e.g., WO 2019/118902, the disclosure of which is incorporated by reference herein in its entirety.

Thus, there remains a need for improved T cell therapy in which the engineered T cells have high as well as sustained tumor-killing potency.

### SUMMARY OF THE INVENTION

The present disclosure provides mammalian (e.g., human) T cells comprising one or more expression vectors encoding c-Jun (e.g., human c-Jun), wherein expression level of Regnase-1 or protein tyrosine phosphatase non-receptor type 2 (PTPN2) in the cell is reduced due to, e.g., a null mutation in the Regnase-1 or PTPN2 gene or due to RNA interference or an antisense oligonucleotide targeting Regnase-1 or PTPN2 mRNA.

In another aspect, the present disclosure provides methods of increasing T cell function, reducing T cell exhaustion, increasing T cell survival, comprising: (A) providing a mammalian (e.g., human) T cell comprising an expression vector for expressing a c-Jun (e.g., a human c-Jun), wherein the T cell overexpresses c-Jun (e.g., human c-Jun) compared to a reference cell without the expression vector, and introducing (i) a null mutation to one or both alleles of the Regnase-1 or PTPN2 gene in the cell, or (ii) an RNA interfering molecule or an antisense oligonucleotide targeting Regnase-1 or PTPN2 mRNA; or (B) providing a mammalian (e.g., human) T cell having reduced expression of Regnase-1 or PTPN2 due to (i) a null mutation to one or both alleles of the Regnase-1 or PTPN2 gene in the cell, or (ii) an RNA interfering molecule or an antisense oligonucleotide targeting Regnase-1 or PTPN2 mRNA, and introducing to the cell an expression vector for expressing c-Jun (e.g., a human c-Jun), wherein the T cell overexpresses c-Jun (e.g., human c-Jun) compared to a reference cell without the expression vector; thereby obtaining a human T cell with increased T cell function, reduced exhaustion, or increased survival.

In some embodiments, the T cell (e.g., human T cell) herein comprises a null mutation on both alleles of the Regnase-1 or PTPN2 gene.

In some embodiments, the T cell (e.g., human T cell) herein further comprises an expression cassette for expressing a recombinant antigen receptor, such as an engineered T cell receptor (TCR) or a chimeric antigen receptor (CAR). In some embodiments, the recombinant antigen receptor is specific for a tumor antigen, optionally selected from CD19, CD20, CD22, ROR1, GD2, an EBV antigen, folate receptor, mesothelin, human carcinoembryonic antigen, CD33/IL3Ra, c-Met, PSMA, Glycolipid F77, EGFRVIII, NY-ESO-1, MAGE-A3, MART-1, GP1000, HER2, BCMA, and a combination thereof. In some embodiments, the T cell comprises a polycistronic (e.g., bi- or tri-cistronic) expression cassette for expressing the human c-Jun and a CAR or an engineered TCR.

In some embodiments, the c-Jun used herein is a wildtype human c-Jun (e.g., SEQ ID NO: 1) or comprises an amino acid sequence at least 90% identical to SEQ ID NO:1. In some embodiments, the c-Jun is a mutant human c-Jun, optionally comprising an inactivating mutation in its transactivation domain or delta domain. In further embodiments, the c-Jun comprises (i) S63A and S73A mutations or (ii) a deletion between residues 2 and 102 or between residues 30 and 50 as compared to wildtype c-Jun.

In some embodiments, the human T cell is CD4⁺ and/or CD8⁺. In some embodiments, the T cell is a tumor-infiltrating lymphocyte (TIL).

Provided herein also are pharmaceutical compositions comprising the present T cells and a pharmaceutically acceptable carrier.

In another aspect, the present disclosure provides a method of treating a subject in need thereof, comprising administering to the subject the present T cells or pharmaceutical compositions. The cells may be, for example, autologous or allogeneic T cells. Also provided herein are cells and pharmaceutical compositions for use in treating a subject in need thereof and use of the cells herein for the manufacture of a medicament for treating a subject in need thereof. In some embodiments, the subject (e.g., a human patient) has cancer (e.g., a solid tumor).

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESRIPTION OF THE DRAWINGS

FIGS. 1A-D are graphs showing the efficacy of ROR1 CAR T cells overexpressing c-Jun in a genetic knockout background in an H1975 xenograft tumor model. High: high T cell dose (1×10⁶ cells) (FIGS. 1A and 1B). Low: low T cell dose (2.5×10⁵ cells) (FIGS. 1C and 1D).

FIGS. 2A and 2B are graphs showing the enumeration of ROR1 CAR T cells in the peripheral blood of H1975 tumor-bearing NSG MHCI/II KO mice. The animals were injected with 1×10⁶ (FIG. 2A) or 2.5×10⁵ (FIG. 2B) ROR1 CAR T cells.

### DETAILED DESCRIPTION OF THE INVENTION

The present disclosure provides engineered human cells (e.g., immune cells such as T cells) comprising expression constructs for overexpressing c-Jun (e.g., a human c-Jun) and optionally a recombinant antigen receptor such as an engineered T cell receptor (TCR) or a chimeric antigen receptor (CAR); these human cells also have reduced Regnase-1 or PTPN2 expression, caused by, e.g., null mutations in the Regnase-1 or PTPN2 gene or introduction of siRNA or antisense agents.

Overexpression of c-Jun in these therapeutic (e.g., T) cells helps sustain the active state of the cells by, e.g., alleviating, reducing, or preventing T cell dysfunction (e.g., T cell exhaustion). The present inventors have unexpectedly found that reduction of Regnase-1 or PTPN2 expression caused by knockout of the corresponding gene further enhance c-Jun's aforementioned activities. The present engineered immune cells such as T cells exhibit sustained, potent cytotoxicity against tumor cells. As compared to T cells that do not overexpress c-Jun (e.g., through an exogenously introduced c-Jun gene sequence) in the Regnase-1 or PTPN2 knockout background, the present engineered T cells display fewer signs of T cell exhaustion. The engineered cells may have one or more of the following characteristics: (i) they do not have increased expression of exhaustion markers PD-1, TIM-3, LAG-3, TIGIT, and/or CD39 over time, (ii) they have reduced rates of apoptosis, (iii) they maintain an active biological state including secretion of cytokines including IL-2 and INF-γ, (iv) they have enhanced cytotoxicity; (v) they display increased recognition of tumor targets with low surface antigen; (vi) they have enhanced proliferation in response to antigen; and (vii) maintain survival and functionality after repeated antigen stimulation.

### I. Immune Cell Sources

The source of the engineered immune cells of the present disclosure may be a patient to be treated (i.e., autologous cells) or from a donor who is not the patient to be treated (e.g., allogeneic cells). In some embodiments, the engineered immune cells are engineered T cells. The engineered T cells herein may be CD4⁺CD8⁺(i.e., CD4 single positive) T cells, CD4⁻CD8⁺ (i.e., CD8 single positive) T cells, or CD4⁺CD8⁺ (double positive) T cells. Functionally, the T cells may be cytotoxic T cells, helper T cells, natural killer T cells, suppressor T cells, or a mixture thereof. The T cells to be engineered may be autologous or allogeneic.

Primary immune cells, including primary T cells, can be obtained from a number of tissue sources, including peripheral blood mononuclear cells (PBMCs), bone marrow, lymph node tissue, cord blood, thymus tissue, tissue from a site of infection, ascites, pleural effusion, spleen tissue, and/or tumor tissue. Leukocytes, including PBMCs, may be isolated from other blood cells by well-known techniques, e.g., FICOLL™ separation and leukapheresis. Leukapheresis products typically contain lymphocytes (including T and B cells), monocytes, granulocytes, and other nucleated white blood cells. T cells are further isolated from other leukocytes, for example, by centrifugation through a PERCOLL™ gradient or by counterflow centrifugal elutriation. A specific subpopulation of T cells, such as CD3″, CD25″, CD28″, CD4″, CD8″, CD45RA″, GITR″, and CD45RO″ T cells, can be further isolated by positive or negative selection techniques (e.g., using fluorescence-based or magnetic-based cell sorting). For example, T cells may be isolated by incubation with any of a variety of commercially available antibody-conjugated beads, such as Dynabeads®, CELLection™, DETACHaBEAD™ (Thermo Fisher) or MACS® cell separation products (Miltenyi Biotec), for a time period sufficient for positive selection of the desired T cells or negative selection for removal of unwanted cells.

In some instances, autologous T cells are obtained from a cancer patient directly following cancer treatment. It has been observed that following certain cancer treatments, in particular those that impair the immune system, the quality of T cells collected shortly after treatment may have an improved ability to expand ex vivo and/or to engraft after being engineered ex vivo.

Whether prior to or after genetic modification, T cells can be activated and expanded generally using methods as described, for example, in U.S. Pat. Nos. 5,858,358; 5,883,223; 6,352,694; 6,534,055; 6,797,514; 6,867,041; 6,692,964; 6,887,466; 6,905,680; 6,905,681; 6,905,874; 7,067,318; 7,144,575; 7,172,869; 7,175,843; 7,232,566; 7,572,631; and 10,786,533. Generally, T cells may be expanded in vitro or ex vivo by contact with a surface having attached thereto an agent that stimulates a CD3/TCR complex associated signal and a ligand that stimulates a costimulatory molecule on the surface of the T cells. In particular, T cell populations may be stimulated, such as by contact with an anti-CD3 antibody or antigen-binding fragment thereof, or an anti-CD3 antibody immobilized on a surface, or by contact with a protein kinase C activator (e.g., bryostatins) in conjunction with a calcium ionophore. For co-stimulation of an accessory molecule on the surface of the T cells, a ligand that binds the accessory molecule may be used. For example, a population of T cells can be contacted with an anti-CD3 antibody and an anti-CD28 antibody under conditions appropriate for stimulating proliferation of the T cells. To stimulate proliferation of either CD4+ T cells or CD8″ T cells, an anti-CD3 antibody and an anti-CD28 antibody may be employed.

The cell culture conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the culture conditions include addition of IL-2, IL-7 and/or IL-15.

In some embodiments, the cells to be engineered may be pluripotent or multipotent cells that are differentiated into mature T cells after engineering. These non-T cells may be allogeneic and may be, for example, human embryonic stem cells, human induced pluripotent stem cells, or hematopoietic stem or progenitor cells. For ease of description, pluripotent and multipotent cells are collectively called “progenitor cells” herein.

In certain embodiments, where allogeneic cells are used, they are engineered to reduce graft-versus-host rejection (e.g., by knocking out the endogenous B2M and/or TRAC genes).

### II. Engineering of Immune or Progenitor Cells

The immune cells (e.g., T cells) or progenitor cells herein are engineered to express an exogenous (i.e., recombinant) antigen receptor and overexpress c-Jun (e.g., a human c-Jun) in the background of Regnase-1 or PTPN2 knockout. The recombinant antigen receptor may bind specifically to a ligand on a tumor cell. As used herein, a receptor (e.g., TCR or CAR) is said to specifically bind to a ligand when the binding has a K_{D} less than or equal to 1 μM and/or has an off-rate (k_{off}) of 1×10⁻³ S⁻¹ or slower, as measured by surface plasmon resonance (using, e.g., a Biacore™ or Octet™ system).

### A. Null Mutations of T Cell Genes

The present engineered T cells have reduced (e.g., no) expression of one or more genes that are normally active in T cells. In some embodiments, the gene encodes Regnase-1 or PTPN2. The reduced expression may be caused by a knockout of one or both alleles of the gene (i.e., a null mutation or gene ablation), or by a temporary knockdown.

Human Regnase-1 (regulatory RNase 1), also known as MCPIP-1, is encoded by the Z (3H12A gene. Regnase-1 is an endoribonuclease involved in various biological functions such as cellular inflammatory response and immune homeostasis, glial differentiation of neuroprogenitor cells, cell death of cardiomyocytes, adipogenesis, and angiogenesis. Regnase-1 functions as an endoribonuclease involved in mRNA decay (see, e.g., Mao et al., Cell Mol Imm. (2017) 14:412-22). The Z (′3H12A gene is located at chromosome 1p34.3, genomic coordinates (GRCh38): 1:37,474,517-37,484,376. This gene can be ablated (knocked out) by a variety of known gene editing techniques. Mutations such as deletions, insertions, and/or point mutations (e.g., base editing) can be introduced into the transcription regulatory regions (e.g., the promoter region; the transcription start region; or the second intron, which contains an IL-1β-responsive region (Mao, supra)), splice sites, and/or one or more of the gene's exons so that a functional Regnase-1 can no longer be expressed from the edited gene.

Human PTPN2 (protein-tyrosine phosphatase non-receptor type 2) is encoded by a gene by the same name at chromosome 18p11.21, genomic coordinates 18:12, 785,477-12,884,350. PTPN2 is a member of the protein tyrosine phosphatase (PTP) family. Members of the PTP family share a highly conserved catalytic motif, which is essential for the catalytic activity. PTPs are known to be signaling molecules that regulate a variety of cellular processes including cell growth, differentiation, mitotic cycle, and oncogenic transformation. The PTPN2 gene can be knocked out by a variety of known gene editing techniques. For example, mutations such as deletions, insertions, and/or point mutations (e.g., base editing) can be introduced into the transcription regulatory regions (e.g., the promoter region or the transcription start region), splice sites, and/or one or more of the gene's exons so that a functional PTPN2 can no longer be expressed from the edited gene.

In some embodiments, the gene editing technique used is CRISPR-based (e.g., CRISPR/Cas9 or CRISPR/cpf1). Guide RNAs used in such gene editing may be designed by software programs such as the Genetic Perturbation Platform provided by the Broad Institute. See, e.g., Wei et al., Nature 576 (7787): 471-6; Wiede et al., EMBO J. (2020) 39:e103637; and Manguso et al., Nature (2017) 547 (7664): 413-8. Other gene editing systems may also be used, such as those utilizing genome-targeting elements including a DNA-binding domain (e.g., zinc finger DNA-binding protein or a TALE DNA-binding domain) and guide DNA elements (e.g., NgAgo guide DNA). Programmable gene-targeting and nuclease elements enable precise genome editing by introducing DNA breaks, such as double-stranded breaks at specific genomic loci. The genome editing system may, for example, be a meganuclease based system, a zinc finger nuclease (ZFN) based system, a Transcription Activator-Like Effector-based Nuclease (TALEN) based system, or NgAgo-based system.

In some embodiments, the gene of interest (e.g., Z (′3H12A and PTPN2) can be knocked down transiently, by antisense oligonucleotides or RNA interference. See, e.g., Wieder, supra.

B. c-Jun

In some embodiments, the c-Jun is a human c-Jun, such as wildtype human c-Jun (c-JunWT) having the following sequence (available at GenBank under accession number AAA59197.1 or at UniProtKB (under accession number P05412.2):

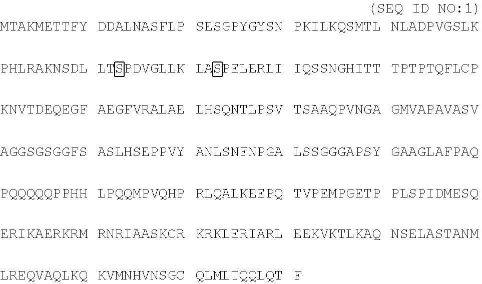

See also Hattori et al., PNAS (1988) 85:9148-52. Alternatively, the c-Jun is a mutant human c-Jun so long as the mutant c-Jun does not impact the mutant's ability to rescue dysfunctional (exhausted) T cells. In some embodiments, a mutant c-Jun comprises at least 70% (e.g., at least 75, 80, 85, 90, 95, or 99%) sequence identity with the C-terminal amino acid residues (e.g., C-terminal 50, 75, 100, 150, 200, or 250 or more residues), the C-terminal portion (e.g., quarter, third, or half) or C-terminal domains (e.g., epsilon, bZIP, and amino acids C-terminal thereof) of a wildtype c-Jun. In some embodiments, the N-terminal amino acid residues (e.g., N-terminal 50, 75, 100, or 150 or more), the N-terminal portion (e.g., quarter, third, or half) or N-terminal domains (e.g., delta, transactivation domain, and amino acids N-terminal thereof) of a wildtype c-Jun are deleted, mutated, or otherwise inactivated.

The percent identity of two amino acid sequences (or of two nucleic acid sequences) may be obtained by, e.g., BLAST® using default parameters (available at the U.S. National Library of Medicine's National Center for Biotechnology Information website). In some embodiments, the length of a reference sequence aligned for comparison purposes is at least 30%, at least 40%, at least 50%, at least 60%, at least 70%, at least 80%, at least 90%, or at least 95% of the reference sequence.

In some embodiments, the c-Jun comprises an inactivating mutation (e.g., substitutions, deletions, or insertions) in its transactivation domain and/or its delta domain. In some embodiments, the c-Jun comprises one or both of S63A and S73A mutations (the positions are boxed above). In some embodiments, the c-Jun has a deletion between residues 2 and 102 or between residues 30 and 50 as compared to wildtype human c-Jun.

Due to introduction of an exogenously introduced c-Jun coding sequence, the engineered T cells overexpress, i.e., express a higher level (e.g., at least 10, 20, 30, 40, 50, 60, 70, 80, 90, or 100% more, or at least 2-, 3-, 4-, 5-, or 10-fold more) of, c-Jun than T cells without such a sequence. In certain embodiments, the engineered T cells express at least about 2-100 fold more, about 5-50 fold more, about 5-40 fold more, about 5-30 fold more, about 5-20 fold more, about 8-20 fold more, or about 10-20 fold more c-Jun than T cells without such a sequence.

### C. Recombinant Antigen Receptors

In some embodiments, the T cells herein express recombinant antigen receptor. As used herein, a “recombinant antigen receptor” refers to an antigen receptor that is not natively expressed by the T cells. A recombinant antigen receptor may be a cell surface molecule that binds to an antigen of interest on another cell (e.g., a tumor cell), and may, for example, be derived from a T cell receptor or an antibody. The recombinant antigen receptor may be, for example, an antibody, an engineered antibody such as an scFv, a CAR, an engineered TCR, a TCR mimic (e.g., an antibody-T cell receptor (abTCR) or a chimeric antibody-T cell receptor (caTCR)), a chimeric signaling receptor (CSR), TCR mimics (e.g., antibodies that recognize epitopes similar to those recognized by TCRs), TCR fusion constructs (TRuCs). See, e.g., EP340793B1, WO 2017/070608, WO 2018/200582, WO 2018/200583, WO 2018/200585, Xu et al., Cell Discovery (2018) 4:62, Baeuerle et al., Nat Comm. (2019) 10:2087.

By way of example, a CAR may comprise an extracellular antigen-binding domain (e.g., a scFv domain), a transmembrane domain, and intracellular signaling domains, optionally peptide stretches linking the domains (e.g., a hinge region linking the antigen-binding domain and the transmembrane domain). In some embodiments, the transmembrane domain may be derived from a natural source, for example, the TCR alpha, beta, gamma, or delta chain, CD3 epsilon, CD4, CD5, CD8, CD9, CD16, CD19, CD20, CD21, CD22, CD25, CD28, CD33, CD37, CD45, CD64, CD80, CD86, CD134, CD137, CD154, or 4-1BB. Alternatively, the transmembrane domain may be synthetic and may comprise predominantly hydrophobic residues (e.g., alanine, leucine, valine, glycine, isoleucine, proline, phenylalanine, and tryptophan). In some embodiments, the intracellular signaling domains are those that provide a signal similar to that from a natural antigen receptor and may comprise, for example, a costimulatory domain (e.g., one derived from CD28, 4-1BB, OX40, DAP10, or ICOS) and a primary signaling domain (e.g., one derived from CD3 zeta chain).

In some embodiments, an abTCR may comprise an engineered TCR in which the antigen-binding domain of a TCR (e.g., an alpha/beta TCR or a gamma/delta TCR) has been replaced by that of an antibody (with or without the antibody's constant domains); the engineered TCR then becomes specific for the antibody's antigen while retaining the TCR's signaling functions.

In some embodiments, a CSR may comprise (1) an extracellular binding domain (e.g., natural/modified receptor extracellular domain, natural/modified ligand extracellular domain, scFv, nanobody, Fab, DARPin, and affibody), (2) a transmembrane domain, and (3) an intracellular signaling domain (e.g., a domain that activates transcription factors, or recruits and/or activates JAK/STAT, kinases, phosphatases, and ubiquitin; SH3; SH2; and PDZ).

The recombinant antigen receptor may target an antigen of interest (e.g., a tumor antigen or an antigen of a pathogen). The antigens may include, without limitation, AFP (alpha-fetoprotein), αvβ6 or another integrin, BCMA, B7-H3, B7-H6, CA9 (carbonic anhydrase 9), CCL-1 (C-C motif chemokine ligand 1), CD5, CD19, CD20, CD21, CD22, CD23, CD24, CD30, CD33, CD38, CD40, CD44, CD44v6, CD44v7/8, CD45, CD47, CD56, CD66e, CD70, CD74, CD79a, CD79b, CD98, CD123, CD138, CD171, CD352, CEA (carcinoembryonic antigen), Claudin 18.2, Claudin 6, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein a), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRC5D (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGFIR (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MCSP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPa (signal-regulatory protein alpha), SLIT, SLITRK6 (NTRK-like protein 6), STEAPI (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens.

In some embodiments, the antigen receptor may be bispecific and target two different antigens, such as two of the antigens listed above. For example, the antigen receptor, such as a CAR, targets CD19 and CD20, or CD19 and CD22.

### D. Nucleic Acids

The recombinant antigen receptor and the c-Jun may be introduced to the T cells or progenitor cells through one or more nucleic acid molecules (e.g., DNA or RNA such as mRNA). In some embodiments, the nucleic acid molecules may be placed on one or more DNA or RNA vectors for introduction into the host cells.

The nucleic acid molecules (e.g., DNA or RNA vectors containing them) may be introduced into the cells by well-known techniques, including without limitation, electroporation, calcium phosphate precipitation, lipofection, particle bombardment, microinjection, colloidal dispersion systems (e.g., as macromolecule complexes, nanocapsules, microspheres, and beads), and lipid-based systems (e.g., oil-in-water emulsions, micelles, mixed micelles, and liposomes). Alternatively, the nucleic acid molecules may be introduced into the cells by transduction of recombinant viruses whose genomes comprise the nucleic acid molecules. Examples of viral vectors include, without limitation, vectors derived from lentivirus, retrovirus, adenovirus, adeno-associated virus, herpes simplex virus, Sendai virus, and vaccinia virus. In certain embodiments, the recombinant virus is pseudotyped with a heterologous envelope protein. In one embodiment, the recombinant virus is a lentivirus pseudotyped with an envelope glycoprotein derived from vesicular stomatitis virus (VSV), measles virus, or another virus (see e.g., Cronin et al., Curr Gene Ther. (2005) 5 (4): 387-98; Gutierrez-Guerrero et al., Viruses (2020) 12 (9): 1016).

In some embodiments, the coding sequences for the antigen receptor polypeptide chains and the c-Jun may be placed on separate expression constructs. In some embodiments, the coding sequences for the antigen receptor and the c-Jun may be placed on a single expression construct. The coding sequences may be placed into one or more expression cassettes on the construct, each cassette being its own transcription unit (e.g., with its own promoter and polyadenylation site and other transcription control elements). In particular embodiments, the coding sequences may be placed into a single expression cassette (e.g., a bi- or tri-cistronic expression cassette), with the coding sequences being transcribed under a common promoter. In a polycistronic arrangement, the coding sequences are in-frame and separated from each other by the coding sequence of a self-cleaving peptide (e.g., a 2A self-cleaving peptide such as a T2A, P2A, E2A, or F2A peptide) and/or a consensus recognition sequence for a Furin protease (see, e.g., Limstra et al., J Virol. (1999) 73 (8): 6299-6306 and Thomas, G., Nat Rev Mol Cell Biol. (2002) 3 (10): 753-66). Alternatively, the coding sequences may be separated from each other by a ribosomal internal entry site (IRES). Thus, the polycistronic (e.g., tri-cistronic) expression cassette is transcribed into a single RNA but ultimately the single RNA is processed and translated into separate polypeptides.

In particular embodiments of a tri-cistronic expression cassette, the coding sequence for c-Jun is separated from the coding sequence of a TCR α chain (or a first chain of a bi-specific CAR) by a 2A-encoding sequence; the coding sequence of the TCR α chain is separated from the coding sequence of a TCR β chain (or a second chain of a bi-specific CAR) optionally by a coding sequence for a furin cleavage consensus sequence and a 2A-coding sequence. In some embodiments, the c-Jun coding sequence precedes the antigen receptor coding sequences in the polycistronic expression cassette. In other embodiments, the c-Jun coding sequence follows or resides between the antigen receptor coding sequences.

The expression cassettes (polycistronic or monocistronic) may contain a promoter that is constitutively active in mammalian (e.g., human or human T) cells. Such promoters include, without limitation, an immediate early cytomegalovirus (CMV) promoter, a simian virus 40 (SV40) early promoter, a human immunodeficiency virus (HIV) long terminal repeat (LTR) promoter, an Epstein-Barr virus immediate early promoter, a Rous sarcoma virus promoter, an elongation factor-1α (EF-1α) promoter, an MND promoter, an actin promoter, a myosin promoter, a hemoglobin promoter, and a creatine kinase promoter. Core or minimal promoters derived from the aforementioned promoters also are contemplated. Alternatively, the expression cassettes may comprise an inducible promoter system. Exemplary inducible promoter systems include, without limitation, hormone-regulated elements, synthetic ligand-regulated elements, ionizing radiation-regulated elements, tetracycline (Tet) systems (e.g., “Tet-Off” and “Tet-On” systems), and NFAT systems (see, e.g., Kallunki et al., Cells (2019) 8 (8): 796; Uchibori et al., Mol Ther Oncolytics. (2018) 12:16-25).

In some embodiments, the expression cassettes also include Kozak sequences, polyadenylation sites, and other elements that facilitate transcription and/or translation of the coding sequences. For example, a woodchuck hepatitis virus post-transcriptional response element (WPRE) or variants thereof may be included at the 3′ untranslated region of the expression cassette.

In the expression cassettes, the transcription/translation regulatory elements such as the promoters, any enhancers, and the like are operably linked to the coding sequences so as to allow efficient expression of the coding sequences and efficient translation of the RNA transcripts.

In certain embodiments, the present disclosure provides a single-vector construct (e.g., a lentiviral vector) comprising a polycistronic expression cassette, comprising a mammalian promoter, a c-Jun coding sequence, coding sequences for the antigen receptor, and a polyadenylation signal sequence. The coding sequences are linked by one or more nucleotide linkers selected from a coding sequence for a self-cleaving peptide (e.g., P2A, T2A, E2A, F2A, or functional equivalents thereof) and a furin cleavage consensus sequence.

The coding sequences in the expression cassettes may be codon-optimized for optimal expression levels in a host cell of interest (e.g., human cells).

The nucleic acid molecules encoding the antigen receptor and the c-Jun may be integrated into the genome of the engineered cells, or remain episomal. The integration may be targeted integration occurring through gene editing (e.g., mediated by CRISPR, TALEN, zinc finger nucleases, and meganucleases).

The engineered cells can be enriched for by positive selection techniques. For example, the cells can be selected for their ability to bind to the target antigen in, e.g., flow cytometry assays. To confirm c-Jun expression, RT-PCR may be performed on the engineered immune (e.g., T) cells. The positive selection may lead to enrichment of recombinant antigen receptor-positive/c-Jun-positive cells in a cell population, where the double positive T cells constitute more than 30, 35, 40, 45,50, 55, 60, 65, 70, 75, 80, 85, 90, or 95% of the total cell population. The engineered cells may be cryopreserved until use.

### E. T Cell Exhaustion

Overexpression of c-Jun in T cells helps sustain the active state of the cells by, e.g., alleviating or preventing T cell dysfunction (e.g., T cell exhaustion). The present engineered immune cells, such as T cells, exhibit sustained, potent cytotoxicity against tumor cells. As compared to T cells that do not overexpress c-Jun, the present engineered T cells display fewer signs of T cell exhaustion and increased signs of persistent effector cells.

In certain embodiments, the cells engineered herein have reduced expression of one or more exhaustion markers, including but not limited to, TIGIT, PD-1, TIM-3, LAG-3, and CD39. Expression of exhaustion markers can be measured in bulk populations by flow cytometry, using bulk RNA-Seq transcriptome analysis. Alternatively, individual cell transcriptome analysis may be carried out using single cell RNA-Seq. In certain embodiments, expression of one or more exhaustion markers in the engineered T cells overexpressing c-Jun is reduced by at least about 1.5, 2, 2.5, 3.0, 3.5, 4, 5, 10, 15, 20, 25, 30, 35, 40, 45, 50, 55, 60, 65, 75, or 100-fold.

In certain embodiments, a population of the present engineered T cells secretes at least about 5, 10, 15, 20, 25, 30, 35, 40, 45, 50, 55, 60, 65, 70, 75, 80, 85, 90, 95, 100, 125, or 150-fold more of IL-2, INF-γ, and/or TNF-α as compared to a control population of engineered T cells that do not overexpress c-Jun. In particular embodiments, a population of the present engineered T cells express at least about 2, 2.5, 3, 3.5, 4, 4.5, 5, 5.5, 6, 8, 10, or 15-fold more of IL-2, INF-γ, and/or TNF-α at day 0 and/or day 14 of persistent antigen stimulation at a 1:1, 1:5, 1:10 or 1:20 E:T ratio, as compared to a control population of engineered T cells that do not overexpress c-Jun. Cytokine secretion can be measured by methods known in the art such as ELISA and Meso Scale Discovery (MSD) analysis.

In certain embodiments, a population of the present engineered T cells demonstrates at least about 2, 4, 6, 8, 10, 15, 20, 25, 30, 40, 50, 75 or 100-fold higher enhanced cytotoxicity efficiency as compared to a control population of engineered CD8⁺ T cells that do not overexpress c-Jun, for example as quantified by area under curve (AUC).

In some embodiments, a population of the present engineered T cells demonstrate about the same, or at least about 1.5, 2, 2.5, 3, 3.5, 4, 5, 8, 10, 15, 20, 25, 30, 50, 75, 100, 125, 150, 200, 225, 250, 300, 400, or 500-fold more enhanced proliferation in response to antigen, as compared to a control population of engineered T cells that do not overexpress c-Jun. Antigen-induced proliferation can be tested by proliferation assays known in the art, such as those described herein.

Assays useful for measuring exhaustion, cell phenotype, persistence, cytotoxicity and/or killing, proliferation, cytokine release, and gene expression profiles are known in the art and include, for example flow cytometry, intracellular cytokine staining (ICS), IncuCyte® immune cell killing analysis, MSD or similar assay, persistent antigen stimulation assay, sequential antigen stimulation assay (similar to persistent antigen stimulation assay but without resetting E:T cell ratio with each round of restimulation), bulk and single cell RNA-seq, cytotoxicity assays, ELISA, Western blot, and other standard molecular and cell biology methods. See, e.g., Geraci et al., Fron Genet. (2020) 11:220; Sturm et al., Bioinformatics (2019) 35 (14): 1436-45; Van den Berge et al., Ann Rev Biomed. (2019) 2:139-73); Current Protocols in Molecular Biology or Current Protocols in Immunology (John Wiley & Sons, Inc., 1999-2021).

### III. Pharmaceutical Compositions and Uses

The present disclosure provides pharmaceutical compositions comprising the engineered T cells described herein. The pharmaceutical compositions may comprise a pharmaceutically acceptable carrier that is suitable to maintain the health of the cells before introduction into the patient.

In some embodiments, engineered cells can be harvested from a culture medium, and washed and concentrated into a carrier in a therapeutically effective amount. Exemplary carriers include saline, buffered saline (e.g., phosphate buffered saline), physiological saline, water, Hanks' solution, Ringer's solution, Nonnosol-R (Abbott Labs), Plasma-Lyte A(R) (Baxter Laboratories, Inc., Morton Grove, IL), glycerol, ethanol, and combinations thereof. It is preferred that the carrier is isotonic. In some embodiments, the carrier can be supplemented with ingredients such as human serum albumin (HSA) or other human serum components, 5% glucose or dextrose. Additional isotonic agents include polyhydric sugar alcohols including trihydric or higher sugar alcohols, such as glycerin, erythritol, arabitol, xylitol, sorbitol, or mannitol may also be included.

The pharmaceutical T cell compositions may be administered in a therapeutically effective amount to a cancer patient systemically (e.g., through intravenous or portal vein injection) or locally (e.g., through intratumoral injection). In some embodiments, the compositions such as those targeting a tumor antigen are used to treat a patient with cancer such as a solid tumor. The cancer may be, without limitation, melanoma (e.g., metastatic melanoma), lung cancer (e.g., non-small cell lung cancer), myeloma (e.g., multiple myeloma), esophageal cancer, synovial sarcoma, gastric cancer, colorectal cancer, breast cancer, liver cancer (e.g., hepatocarcinoma), head and neck cancer, ovarian cancer, prostate cancer, urothelial cancer, and bladder cancer. As used herein, the term “treatment” or “treating” refers to an approach for obtaining beneficial or desired results in the treated subject. Such results include, but are not limited to: alleviating one or more symptoms resulting from the disease, diminishing the extent of the disease (e.g., reducing tumor volumes), stabilizing the disease (e.g., preventing or delaying the worsening of the disease), preventing or delaying the spread (e.g., metastasis) of the disease, preventing or delaying the recurrence or relapse of the disease, ameliorating the disease state, providing a remission (partial or total) of the disease, decreasing the dose of one or more other medications required to treat the disease, improving the quality of life, restoring body weight, and/or extension of survival (e.g., overall survival or progression-free survival).

A therapeutically effective amount of the composition refers to the number of engineered T cells sufficient to achieve a desired clinical endpoint. In some embodiments, a therapeutically effective amount contains more than 10⁴, 10⁵, 10⁶, 10⁷, 10⁸, or 10⁹ of the engineered cells. In certain embodiments, a subject is administered with a range of about 10⁶-10¹¹ engineered cells.

The pharmaceutical composition in some embodiments comprises the cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells and compositions in some embodiments are administered using standard administration techniques, formulations, and/or devices. Provided are formulations and devices, such as syringes and vials, for storage and administration of the compositions. Administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition of the present disclosure (e.g., a pharmaceutical composition containing a genetically modified cell), it will generally be formulated in a unit dosage injectable form (solution, suspension, emulsion).

In one aspect, the present disclosure provides pharmaceutical compositions comprising the nucleic acid molecules for expressing the recombinant antigen receptor (e.g., TCR or CAR) and c-Jun. The nucleic acid molecules may be as described above, such as the viral vectors (e.g., lentiviral vectors) described above. The pharmaceutical compositions are used ex vivo to engineer T or progenitor cells, which are then introduced to the patient. The pharmaceutical compositions comprise the nucleic acid molecules or the recombinant viruses whose genome comprise the expression cassettes for the recombinant antigen receptor (e.g., TCR or CAR) and c-Jun and a pharmaceutically acceptable carrier such as a buffered solution that optionally comprises other agents such as preservatives, stabilizing agents, and the like.

The pharmaceutical compositions may be provided as articles of manufacture, such as kits, that include vials (e.g., single-dose vials) comprising the biological materials (the cells or the nucleic acid molecules or recombinant viruses) and optionally instructions for use.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of immunology, medicine, medicinal and pharmaceutical chemistry, and cell biology described herein are those well-known and commonly used in the art. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### EXAMPLES

In the Examples provided below, we studied the effects of four genes on the activation state of CAR T cells in a tumor microenvironment (TME). These four genes, VHL, XBP 1, Regnase 1, and PTPN2, had been associated with T cell dysfunction and blunted anti-tumor response. (VHL: Phan et al., Immunity (2016) 45:1024-37); XBP1: Ma et al., Cell metabolism (2019) 30:143-156.e5; Song et al., Nature (2018) 562:423-8); and Regnase-1 and PTPN2: Wei et al., Nature (2019) 576:471-6). We tested whether the knockout of these genes would enhance the anti-tumor activity of CAR T cells in combination with c-Jun overexpression.

The T cells used in these studies were T cells carrying an expression construct for anti-ROR1 CAR with or without also a coding sequence for human c-Jun. VHL, XBP1, Regnase 1, or PTPN2 was knocked out by Cas9 ribonucleoprotein (RNP) (Synthego) delivered into the cells by electroporation. The effect of the knockout on CAR T function was compared to cells without the knockout. The in vitro results were then validated by using a H1975 xenograft model.

### Materials and Methods

### Lentiviral Constructs

Lentiviral vector (LVV) constructs were generated with an expression cassette containing the following: the coding sequences for (i) a human wildtype c-Jun or truncated human CD19, (ii) a ROR1 CAR, and (iii) a truncated version of human EGFR. The ROR1 CAR was derived from the R12 anti-ROR1 antibody (Yang et al., PLOS One (2011) 6:e21018) and contains a CD28-derived transmembrane domain, a 4-1BB costimulatory domain, and a CD3 zeta signaling domain.

### T Cell Production

For viral construct transduction, gene editing, and cell expansion, T cells were activated on Day 0, transduced with viral constructs on Day 1, electroporated with Cas9 RNP on Day 2, and expanded from Day 3. On Day 7, the cells were analyzed for CAR transduction efficiency and genomic editing efficiency (T7).

Briefly, T cell products were generated using CD4″ and CD8″ cells isolated from peripheral blood mononuclear cells from three healthy donors. On day 0, T cells were thawed and mixed at CD4:CD8 ratio of 1:1 in T Cell Medium (TCM) consisting of OpTmizer™ medium, OpTmizer™ Supplement (Thermo Fisher), supplemented with CTS™ Immune Cell SR (Thermo Fisher), 2 mM L-Glutamine (Gibco), 2 mM GlutaMAX™ (Thermo Fisher), 200 IU/mL human IL-2 (R&D Systems), 1200 IU/mL human IL-7 (R&D Systems), and 200 IU/mL human IL-15 (R&D Systems). T cells were activated using 1% TransAct™ (Miltenyi).

On day 1, T cells were transduced in 24-well plates with lentiviral vectors (LVV) at a multiplicity of infection (MOI) of 5 using lentiviral vectors (LVV) in the presence of 1% LentiBOOST® (Sirion Biotech) at a cell density of 3 million cells per well. Transduction was continued in humidified incubators at 37° C. overnight.

On day 2, T cells were electroporated with CRISPR/Cas9 gRNA RNP complexes using a P3 Primary Cell 4D Nucleofector kit (Lonza). For RNP complex formation, the Synthego Gene Knockout Kit v2 was used according to the manufacturer's instructions. Guide RNAs were mixed with SpyFi Cas9 at a ratio of 7.5:1 in P3 electroporation (EP) solution and incubated for at least 10 minutes at room temperature. T cells were resuspended in EP at a concentration of 100 million T cells per mL and 50 μL cell suspension was mixed 1:1 with 50 μL EP containing 2 μM RNP complexes. T cells were electroporated using program DN100 followed by addition of 500 μL TCM medium before centrifugation and resuspension in fresh TCM and seeding into 24-well plates at a density of 2.5 million T cells per well.

On day 3, T cells were transferred into gas-permeable 24-well plates with TCM media and expanded until day 7 after activation and then cryopreserved in CryoStor CS10 (Stemcell Technologies).

Gene knockout was analyzed using T7 analysis. Band intensity served as surrogate for intact genes. Across three donors, gene editing efficiency was greater than 85% for VHL, XBP1 and PTPN2. Regnase−1 knockout efficiency was approximately 50%. Expression of c-Jun expression did not affect knockout efficiency.

### Example 1: Knockout of PTPN2 or Regnase-1 in Combination with c-Jun Overexpression Enhances T Cell Functions

To evaluate the impact of genetic knockouts of VHL, XBP1, Regnase-1, and PTPN2 in combination with c-Jun overexpression on ROR1 CAR T cell function, T cells derived from a healthy donor were tested in a ROR1-expressing H1975 xenograft tumor model in NSG mice. Briefly, tumor cells (i.e., ROR1-positive H1975 NSCLC cell line) were implanted subcutaneously into the flank of NSG-MHC I/II DKO; NOD.Cg-Prkde^{scid} H2-K1^{tmlBpe} H2-Ab1^{emlMvw} H2-1) 1^{tm1Bpe} 112rg^{tm1Wjl}/SzJ mice. When the tumor volume reached about 100 mm³, mice were intravenously infused with one of the following: (i) 1×10⁶ un-transduced mock T cells, (ii) 1×10⁶ control ROR1 CAR T cells (not overexpressing c-Jun), (iii) 1×10⁶ or 2.5×10⁵ ROR1 CAR T cells overexpressing c-Jun, or (iv) 1×10⁶ or 2.5×10⁵ ROR1 CAR T cells overexpression in-Jun in combination with a knockout of VHL, XBP1, Regnase-1, or PTPN2. Anti-tumor activity was assessed by measuring tumor volume using calipers. Treatment-related toxicity was assessed by measuring animal body weight. Additionally, expansion of the T cells was assessed using flow cytometry of peripheral blood.

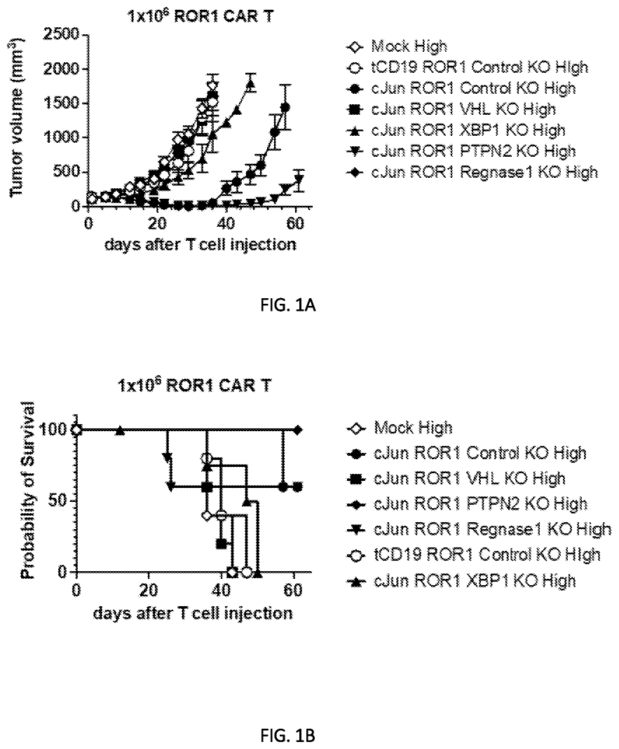

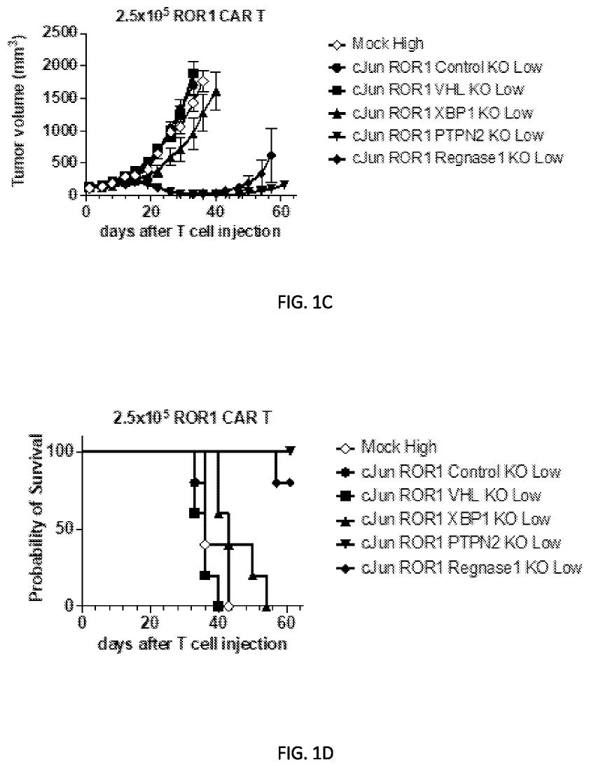

The data show that at a dose of 1×10⁶ CAR T cells, control ROR1 CAR T cells were ineffective at reducing tumor burden or extending survival (FIGS. 1A and 1B). ROR1 CAR T cells overexpressing c-Jun significantly reduced tumor burden and extended survival. ROR1 CAR T cells overexpressing c-Jun in a VHL, or XBP1 knockout background lost the ability to control tumor growth and did not extend animal survival (FIGS. 1A and 1B). Some mice receiving ROR1 CAR T cells overexpressing c-Jun in a Regnase-1 knockout background exhibited body weight loss and had to be taken off study, all surviving animals were able to control tumor growth (FIG. 1A). ROR1 CAR T cells overexpressing c-Jun in a PTPN2 knockout background had prolonged anti-tumor activity without any signs of toxicity (FIG. 1A).

At a dose of 2.5×10⁵ CAR T cells, ROR1 CAR T cells overexpressing c-Jun in a VHL and XBP1 knockout background did not reduce tumor volume or extend animal survival. ROR1 CAR T cells overexpressing c-Jun in a Regnase-1 knockout background significantly reduced tumor volumes and extended animal survival (FIGS. 1C and 1D). ROR1 CAR T cells overexpressing c-Jun in a PTPN2 knockout background had the strongest anti-tumor activity and extended animal survival (FIGS. 1C and 1D).

### Example 2: Knockout of PTPN2 or Regnase-1 in Combination with c-Jun Overexpression Promotes Proliferation of ROR1 CAR T Cells

The persistence of the CAR T cells in the H1795 tumor-bearing NSG mice was evaluated. The animals received a single intravenous administration of one of the following: (i) 1×10⁶ mock (un-transduced) T cells, (ii) 1×10⁶ control ROR1 CAR T cells (i.e., not overexpressing c-Jun), (iii) 1×10⁶ or 2.5×10⁵ ROR1 CAR T cells overexpressing c-Jun, and (iv) 1×10⁶ or 2.5×10⁵ ROR1 CAR T cells overexpressing c-Jun in combination with knockout of VHL, XBP1, Regnase-1, or PTPN2. Then, at various time points post-administration, peripheral blood was collected and the number of CAR T cells per mL of blood was quantified using flow cytometry.

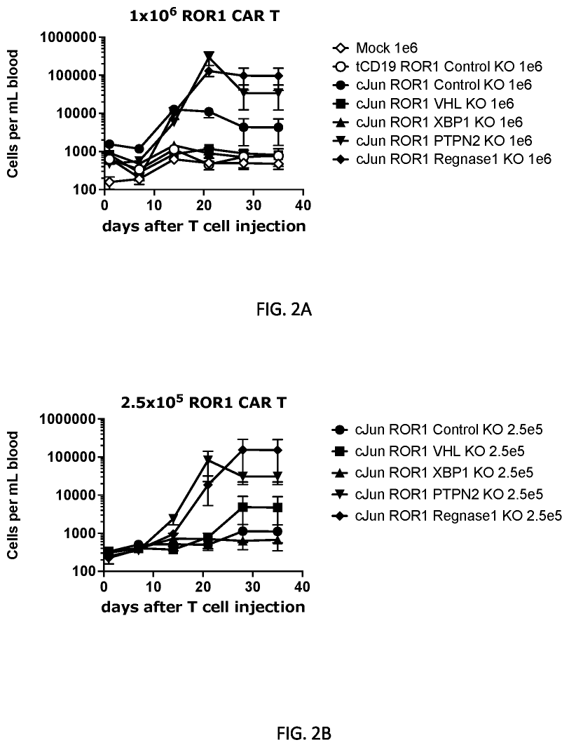

The data show that at a dose of 1×10⁶ CAR T cells, control ROR1 CAR T cells were unable to expand. ROR1 CAR T cells overexpressing c-Jun increased in number by day 14 and persisted at elevated numbers until day 35 (FIG. 2A). ROR1 CAR T cells overexpressing c-Jun in a VHL, or XBP1 knockout background lost the ability to expand (FIG. 2A). ROR1 CAR T cells overexpression c-Jun in a Regnase-1 or PTPN2 knockout background demonstrated an increased ability to expand and reached a maximum at day 21 and then persisted at high numbers until day 35 (FIG. 2A).

At a dose of 2.5×10⁵ CAR T cells, ROR1 CAR T cells overexpressing c-Jun and ROR1 CAR T cells overexpressing c-Jun in a VHL or XBP1 knockout background were unable to expand in number (FIG. 2B). By contrast, ROR1 CAR T cells overexpressing c-Jun in a Regnase-1 or PTPN2 knockout background significantly expanded in number, with cells have PTPN2 knockout reaching a maximum at day 21, and cells with Regnase-1 knockout reached a maximum at day 28. Both T cell products persisted at high numbers until day 35 (FIG. 2B).

Taken together, the above studies show that PTPN2 or Regnase-1 knockout significantly enhance the ability of c-Jun ROR1 CAR T cells to clear tumors. In addition, the Regnase-1 and PTPN2 knockout data indicate that the engineered T cells have significantly higher PK compared to ROR1 CAR T cells that overexpress c-Jun, suggesting that the effect of the Regnase-1 and PTPN2 knockout is related to T cell proliferation. In conclusion, the results demonstrate the unexpected and synergistic effects of PTPN2 or Regnase-1 knockout and c-Jun overexpression in potentiating CAR T functions.

1. A human T cell comprising one or more expression vectors encoding human c-Jun, wherein expression level of Regnase-1 or protein tyrosine phosphatase non-receptor type 2 (PTPN2) in the cell is reduced due to a null mutation in the Regnase-1 or PTPN2 gene or due to RNA interference or an antisense oligonucleotide targeting Regnase-1 or PTPN2 mRNA.

2. A method of increasing T cell function, reducing T cell exhaustion, increasing T cell survival, comprising:
(A)
providing a human T cell comprising an expression vector for expressing a human c-Jun, wherein the T cell overexpresses human c-Jun compared to a reference cell without the expression vector, and
introducing (i) a null mutation to one or both alleles of the Regnase-1 or PTPN2 gene in the cell, or (ii) an RNA interfering molecule or an antisense oligonucleotide targeting Regnase-1 or PTPN2 mRNA; or
(B)
providing a human T cell having reduced expression of Regnase-1 or PTPN2 due to (i) a null mutation to one or both alleles of the Regnase-1 or PTPN2 gene in the cell, or (ii) an RNA interfering molecule or an antisense oligonucleotide targeting Regnase-1 or PTPN2 mRNA, and
introducing to the human T cell an expression vector for expressing a human c-Jun, wherein the T cell overexpresses human c-Jun compared to a reference cell without the expression vector;
thereby obtaining a human T cell with increased T cell function, reduced exhaustion, or increased survival.

3. The cell of claim 1 or method of claim 2, wherein the human T cell comprises a null mutation on both alleles of the Regnase-1 or PTPN2 gene.

4. The cell or method of any one of claims 1-3, wherein the null mutation is generated by CRISPR/Cas9 gene editing.

5. The cell or method of any one of claims 1-4, wherein the human T cell further comprises an expression cassette for expressing a recombinant antigen receptor.

6. The cell or method of claim 5, wherein the recombinant antigen receptor is an engineered T cell receptor (TCR) or a chimeric antigen receptor (CAR).

7. The cell or method of claim 5 or 6, wherein the recombinant antigen receptor is specific for a tumor antigen, optionally selected from CD19, CD20, CD22, ROR1, GD2, an EBV antigen, folate receptor, mesothelin, human carcinoembryonic antigen, CD33/IL3Ra, c-Met, PSMA, Glycolipid F77, EGFRVIII, NY-ESO-1, MAGE-A3, MART-1, GP1000, HER2, BCMA, and a combination thereof.

8. The cell or method of any one of claims 5-7, wherein the T cell comprises a bi-cistronic expression cassette for expressing the human c-Jun and a CAR.

9. The cell or method of any one of claims 5-7, wherein the T cell comprises a tri-cistronic expression cassette for expressing the human c-Jun and the alpha and beta chains, or gamma and delta chains, of an engineered TCR.

10. The cell or method of any one of claims 1-9, wherein the c-Jun is a wildtype human c-Jun, or comprises SEQ ID NO: 1 or an amino acid sequence at least 90% identical thereto.

11. The cell or method of any one of claims 1-9, wherein the c-Jun is a mutant human c-Jun, optionally comprising an inactivating mutation in its transactivation domain or delta domain.

12. The cell or method of claim 11, wherein the c-Jun comprises (i) S63A and S73A mutations or (ii) a deletion between residues 2 and 102 or between residues 30 and 50 as compared to wildtype c-Jun.

13. The cell or method of any one of claims 1-12, wherein the human T cell is a CD4⁺ T cell.

14. The cell or method of any one of claims 1-12, wherein the human T cell is a CD8⁺ T cell.

15. The cell or method of any one of claims 1-12, wherein the human T cell is tumor-infiltrating lymphocyte.

16. A pharmaceutical composition comprising the cell of any one of claims 1 and 3-15 and a pharmaceutically acceptable carrier.

17. A method of treating a subject in need thereof, comprising administering to the subject the cell of any one of claims 1 and 3-15.

18. The method of claim 17, wherein the cell is an autologous or allogeneic T cell.

19. The method of claim 17 or 18, wherein the subject has cancer.

20. The cell of any one of claims 1 and 3-15 for use in treating a subject in need thereof, optionally in a method of any one of claims 17-19.

21. Use of the cell of any one of claims 1 and 3-15 for the manufacture of a medicament for treating a subject in need thereof, optionally in a method of any one of claims 17-19.


SEQUENCE LISTING (1 sequence)

SEQ ID NO: 1 - protein, 331 aa, Homo sapiens
  REGION 1..331: note = misc_feature - wildtype human c-Jun (c-JunWT)
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF